(19) United States
(12) Patent Application Publication
(10) Pub. No.: US 2017/0073724 A1
(43) Pub. Date: Mar. 16, 2017

(54) Method of identifying whether Azotobacter secrets ammonia using nitrogen-free solid incubation media

(71) Applicant: Dongguan Baode Biological Engineering Co., LTD., Dongguan City (CN)

(72) Inventors: Guoping YANG, Dongguan City (CN); Xusheng SUN, Dongguan City (CN); Yajun WANG, Dongguan City (CN); Panpan YANG, Dongguan City (CN); Kun YIN, Dongguan City (CN); Hongbin ZHANG, Dongguan City (CN)

(21) Appl. No.: 15/138,640

(22) Filed: Apr. 26, 2016

(30) Foreign Application Priority Data
Sep. 16, 2015 (CN) 201510588767.5

(51) Int. Cl. C12Q 1/10

(52) U.S. Cl. CPC C12Q 1/10; G01N 2333/245; G01N 2333/21

(57) ABSTRACT

This invention relates to the field of medium and nitrogen fixation technique. More specifically, the present invention relates to a method of identifying whether Azotobacter secrets ammonia using nitrogen-free solid incubation media. Each liter of nitrogen-free incubation media comprises: K₂HPO₄ 0.5-2 g, MgSO₄.7H₂O 0.1-0.5 g, CaCl₂.2H₂O 0.05-0.15 g, glucose 7-13 g, FeSO₄.7H₂O 0.005-0.02 g, NaMoO₄.2H₂O, 0.005-0.01 g, agar 10-20 g, and distilled water to balance. The present invention provides a method to identify whether Azotobacter secrets ammonia using nitrogen-free solid incubation media, which is simple and easy to carry out, and can accurately identify whether Azotobacter secreting nitrogenous compounds to extracellular and the ability of secretion, which play an important role in the development of bio-fertilizer industry.

The current application claims a foreign priority to application number 201510588767.5 filed on Sep. 16, 2015 in China.

### FIELD OF THE INVENTION

This invention relates to the field of medium and nitrogen fixation technique. More specifically, the present invention relates to a method of identifying whether Azotobacter secrets ammonia using nitrogen-free solid incubation media.

### BACKGROUND OF THE INVENTION

A handful of prokaryote organisms has the biological characteristic of nitrogen fixation. These organisms can be divided into two groups: symbiotic nitrogen fixation bacteria and self nitrogen fixation bacteria.

Symbiotic nitrogen fixation bacteria enter the body of higher plants and live with them together. Higher plants as the host of symbiotic nitrogen fixation bacteria provide energy to bacteria. Symbiotic nitrogen fixation bacteria convert nitrogen into ammonia, and give host ammonia in return. Generally, such bacteria do not fix nitrogen in vitro. And they usually fix nitrogen in nothing but leguminous plants.

Self nitrogen fixation bacteria can fix nitrogen in the process of growth without entering into the body of plants. They can fix nitrogen in the soil, in plant rhizosphere or on the plant roots. Thus, self nitrogen fixation bacteria can fix nitrogen in any plant rhizosphere. Besides the capacity of self nitrogen fixation, the capacity of secreting nitrogen nutrition to the outside of the cell (i.e., the ability of secreting ammonia) is also worth considering.

In the process of microbial fertilizer production with self nitrogen fixation bacteria, the capacity of plant promotion is weak when nitrogen nutrition can not be secreted to the extracellular. The greatest challenge of microbial fertilizer production is how to identify the truth Azotobacteria which can secrete nitrogen nutrition to the extracellular.

### DESCRIPTION OF THE INVENTION

The present invention overcomes these and other problems in the art by providing a method of identifying whether Azotobacter secrets ammonia using nitrogen-free solid incubation media, which is simple and easy to carry out, and can accurately identify whether Azotobacter secreting nitrogenous compounds to extracellular A method of identifying whether Azotobacter secrets ammonia using nitrogen-free solid incubation media, which comprising the steps of:

(1) mixing K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media; wherein, each liter of nitrogen-free incubation media comprises: K₂HPO₄ 0.5-2 g, MgSO₄.7H₂O 0.1-0.5 g, CaCl₂.2H₂O 0.05-0.15 g, glucose 7-13 g, FeSO₄.7H₂O 0.005-0.02 g, NaMoO₄.2H₂O, 0.005-0.01 g, agar 10-20 g, and distilled water to balance;

(2) the preparation of double-layer plates are by sterilizing said nitrogen-free incubation media in said step 1 at 118-124° C. for 15-25 minutes, then adding 1 mL indicator bacteria solution into every 100 mL nitrogen-free incubation media when the temperature dropped to 46-48° C. after sterilization, and then pouring nitrogen-free incubation media containing indicator bacteria immediately into sterile petri dish and mixing them, followed by controlling thickness of nitrogen-free incubation media at 2.2-2.8 mm, then pouring water agar of 0.5-1 mm thickness into solidifying nitrogen-free incubation media, as-obtained double-layer plates comprise nitrogen-free solid incubation media containing bacterial of lower layer and sterile water agar of upper layer;

(3) inoculating bacteria to be tested on said nitrogen-free solid incubation media from said step 2, and then placing in incubator at 30° C. for 2-5 days; (4) checking whether indicator bacteria of lower layer of double-layer plates has grown, if indicator bacteria growth occurs, measured Azotobacter does secrete ammonia to extracellular.

Wherein said water agar is in said step 2 is prepared by adding 15 g agar into 1 L of distilled water; then sterilizing at 121° C. for 20 minutes, and placing under incubator at 50° C. for future using.

Wherein said indicator bacteria in said step 2 is Escherichia coli, some microorganisms that are unable to nitrogen fixation can also be used.

The viable concentration of indicator bacteria solution in said step 2 is 20 million/ml. The indicator bacteria solution means aqueous solution containing indicator bacteria.

To avoid the influence of remaining nitrogen source in the test, K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.

Medium be used in this invention is nitrogen-free solid incubation media without nitrogen source, where the bacteria that can not to be nitrogen fixation by self is unviable. The specific method is mixing bacteria that can not fix nitrogen, such as E. coli, as indicator bacteria, with nitrogen-free solid incubation media, afterwards inoculating test bacteria and detecting them in the plates. If the test bacteria secrete ammonia to the outside of the cells, E. coli can grow around the test bacteria. The number of colonies decide the capacity of secreting ammonia.

The present invention provides a method to identify whether Azotobacter secrets ammonia using nitrogen-free solid incubation media, which is simple and easy to carry out, and can accurately identify whether Azotobacter secreting nitrogenous compounds to extracellular and the ability of secretion, which play an important role in the development of bio-fertilizer industry.

### BRIEF DESCRIPTION OF THE DRAWINGS

FIG. 1: Picture of indicator bacteria growing around Azotobacter with ability of secreting ammonia.

FIG. 2: Comparison of indicator bacteria growing around Azotobacter with ability of secreting ammonia and Azotobacter without ability of secreting ammonia.

### DESCRIPTION OF THE PREFERRED EMBODIMENTS

In order to understanding, the following examples and the accompanying drawings are prepared for further illustration. The embodiment of the examples mentioned is not limited to the present invention.

### Example 1

A kind of nitrogen-free incubation media consisting of the following materials in each liter:

| | | |
|---|---|---|
| K₂HPO₄ | 0.5 | g |
| MgSO₄•7H₂O | 0.1 | g |
| CaCl₂•2H₂O | 0.05 | g |
| Glucose | 7 | g |
| FeSO₄•7H₂O | 0.005 | g |
| NaMoO₄•2H₂O | 0.005 | g |
| Agar | 10 | g |

Distilled water to balance.

A method of identifying whether Azotobacter secrets ammonia using nitrogen-free solid incubation media, which comprising the steps of:

(1) mixing above K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media;

(2) the preparation of double-layer plates are by sterilizing said nitrogen-free incubation media in said step 1 at 118° C. for 15 minutes, then adding 1 mL indicator bacteria solution into every 100 mL nitrogen-free incubation media when the temperature dropped to 46° C. after sterilization, and then pouring nitrogen-free incubation media containing indicator bacteria immediately into sterile petri dish and mixing them, followed by controlling thickness of nitrogen-free incubation media at 2.2 mm, then pouring water agar of 0.5 mm thickness into solidifying nitrogen-free incubation media, as-obtained double-layer plates comprise nitrogen-free solid incubation media containing bacterial of lower layer and sterile water agar of upper layer;

(3) inoculating bacteria to be tested on said nitrogen-free solid incubation media from said step 2, and then placing in incubator at 30° C. for 2-5 days;

(4) checking whether indicator bacteria of lower layer of double-layer plates has grown, if indicator bacteria growth occurs, measured Azotobacter does secrete ammonia to extracellular.

Wherein said water agar is in said step 2 is prepared by adding 15 g agar into 1 L of distilled water; then sterilizing at 121° C. for 20 minutes, and placing under incubator at 50° C. for future using.

Wherein said indicator bacteria in said step 2 is Escherichia coli. To avoid the influence of remaining nitrogen source in the test, K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.

### Example 2

A kind of nitrogen-free incubation media consisting of the following materials in each liter:

| | | |
|---|---|---|
| K₂HPO₄ | 1 | g |
| MgSO₄•7H₂O | 0.2 | g |
| CaCl₂•2H₂O | 0.1 | g |
| Glucose | 10 | g |
| FeSO₄•7H₂O | 0.01 | g |
| NaMoO₄•2H₂O | 0.008 | g |
| Agar | 15 | g |

Distilled water to balance.

A method of identifying whether Azotobacter secrets ammonia using nitrogen-free solid incubation media, which comprising the steps of:

(1) mixing above K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media;

(2) the preparation of double-layer plates are by sterilizing said nitrogen-free incubation media in said step 1 at 121° C. for 20 minutes, then adding 1 mL indicator bacteria solution into every 100 mL nitrogen-free incubation media when the temperature dropped to 47° C. after sterilization, and then pouring nitrogen-free incubation media containing indicator bacteria immediately into sterile petri dish and mixing them, followed by controlling thickness of nitrogen-free incubation media at 2.5 mm, then pouring water agar of 0.8 mm thickness into solidifying nitrogen-free incubation media, as-obtained double-layer plates comprise nitrogen-free solid incubation media containing bacterial of lower layer and sterile water agar of upper layer;

(3) inoculating bacteria to be tested on said nitrogen-free solid incubation media from said step 2, and then placing in incubator at 30° C. for 2-5 days;

(4) checking whether indicator bacteria of lower layer of double-layer plates has grown, if indicator bacteria growth occurs, measured Azotobacter does secrete ammonia to extracellular.

Wherein said water agar is in said step 2 is prepared by adding 15 g agar into 1 L of distilled water; then sterilizing at 121° C. for 20 minutes, and placing under incubator at 50° C. for future using.

Wherein said indicator bacteria in said step 2 is Escherichia coli.

To avoid the influence of remaining nitrogen source in the test, K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.

### Example 3

A kind of nitrogen-free incubation media consisting of the following materials in each liter:

| | | |
|---|---|---|
| K2HPO₄ | 1.5 | g |
| MgSO₄•7H₂O | 0.3 | g |
| CaCl₂•2H₂O | 0.1 | g |
| Glucose | 12 | g |
| FeSO₄•7H₂O | 0.01 | g |
| NaMoO₄•2H₂O | 0.0051 | g |
| Agar | 16 | g |

Distilled water to balance.

A method of identifying whether Azotobacter secrets ammonia using nitrogen-free solid incubation media, which comprising the steps of:

(1) mixing above K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media;

(2) the preparation of double-layer plates are by sterilizing said nitrogen-free incubation media in said step 1 at 118-124° C. for 25 minutes, then adding 1 mL indicator bacteria solution into every 100 mL nitrogen-free incubation media when the temperature dropped to 47° C. after sterilization, and then pouring nitrogen-free incubation media containing indicator bacteria immediately into sterile petri dish and mixing them, followed by controlling thickness of nitrogen-free incubation media at 2.6 mm, then pouring water agar of 0.6 mm thickness into solidifying nitrogen-free incubation media, as-obtained double-layer plates comprise nitrogen-free solid incubation media containing bacterial of lower layer and sterile water agar of upper layer;

(3) inoculating bacteria to be tested on said nitrogen-free solid incubation media from said step 2, and then placing in incubator at 30° C. for 2-5 days;

(4) checking whether indicator bacteria of lower layer of double-layer plates has grown, if indicator bacteria growth occurs, measured Azotobacter does secrete ammonia to extracellular.

Wherein said water agar is in said step 2 is prepared by adding 15 g agar into 1 L of distilled water; then sterilizing at 121° C. for 20 minutes, and placing under incubator at 50° C. for future using.

Wherein said indicator bacteria in said step 2 is Escherichia coli.

To avoid the influence of remaining nitrogen source in the test, K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.

### Example 4

A kind of nitrogen-free incubation media consisting of the following materials each liter:

| | | |
|---|---|---|
| K₂HPO₄ | 2 | g |
| MgSO₄•7H₂O | 0.5 | g |
| CaCl₂•2H₂O | 0.15 | g |
| Glucose | 13 | g |
| FeSO₄•7H₂O | 0.02 | g |
| NaMoO₄•2H₂O | 0.01 | g |
| Agar | 20 | g |

Distilled water to balance.

A method of identifying whether Azotobacter secrets ammonia using nitrogen-free solid incubation media, which comprising the steps of: (1) mixing above K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media;

(2) the preparation of double-layer plates are by sterilizing said nitrogen-free incubation media in said step 1 at 124° C. for 25 minutes, then adding 1 mL indicator bacteria solution into every 100 mL nitrogen-free incubation media when the temperature dropped to 48° C. after sterilization, and then pouring nitrogen-free incubation media containing indicator bacteria immediately into sterile petri dish and mixing them, followed by controlling thickness of nitrogen-free incubation media at 2.8 mm, then pouring water agar of 1 mm thickness into solidifying nitrogen-free incubation media, as-obtained double-layer plates comprise nitrogen-free solid incubation media containing bacterial of lower layer and sterile water agar of upper layer;

(3) inoculating bacteria to be tested on said nitrogen-free solid incubation media from said step 2, and then placing in incubator at 30° C. for 2-5 days;

(4) checking whether indicator bacteria of lower layer of double-layer plates has grown, if indicator bacteria growth occurs, measured Azotobacter does secrete ammonia to extracellular.

Wherein said water agar is in said step 2 is prepared by adding 15 g agar into 1 L of distilled water; then sterilizing at 121° C. for 20 minutes, and placing under incubator at 50° C. for future using.

Wherein said indicator bacteria in said step 2 is Escherichia coli.

To avoid the influence of remaining nitrogen source in the test, K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.

### Experimental Data

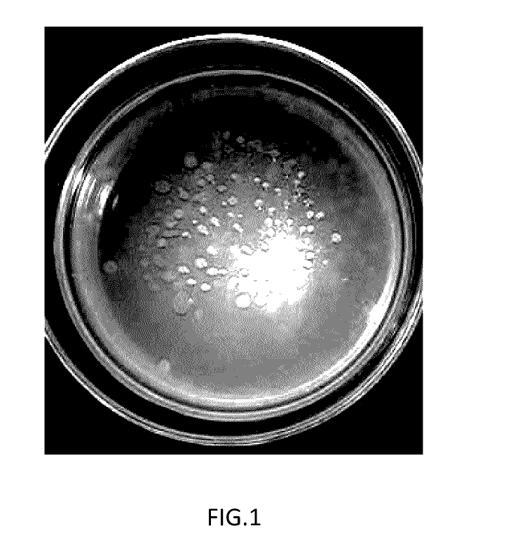

According to the examples from 1 to 4 in this invention, 200 different strains were tested and 72 were detected can induce indicator bacteria grow in varying degrees refer to FIG. 1. It was also found that these 72 strains can promote plant growth in varying degrees, which showed these 72 strains have different capacities of secreting ammonia.

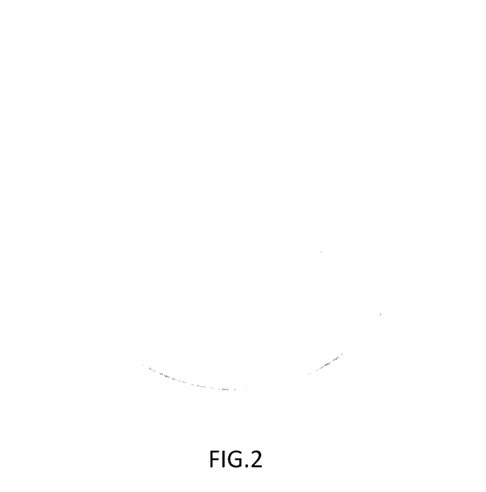

As we can see in FIG. 2, the first is an indicator bacteria that can not fix nitrogen, the colony diameter of indicator bacteria is zero. The second is an indicator bacteria that can fix nitrogen to a certain extent, the colony diameter of indicator bacteria is longer. The third is an indicator bacteria that can fix nitrogen to a larger extent, the colony diameter of indicator bacteria is longest. Furthermore, indicator bacteria with stronger ability to fix nitrogen has the longer diameter.

Next, these 72 strains were assayed for nitrogenase activity, the results showed 68 strains were observed in varying degrees activity. Furthermore, the nitrogenase activity was related to the diameter of colony, the bacteria, which has the longer diameter, has higher nitrogenase activity, and can produce more ammonia to the outside of cells, as shown in table 1.

TABLE 1

| | | | The colony | Activity of |
|---|---|---|---|---|
| | | | diameter of | nitrogenase |
| | | | indicator | [nmol/ |
| No. | Strain | Generic name | bacterium (mm) | (h · mL)] |
| 1 | g1 | Paenibacillus | 3.52 | 1111.362172 |
| | | azotofixans | | |
| 2 | g7 | Bacillus | 3.46 | 1012.258038 |
| | | subtilis | | |
| 3 | t131 | Bacillus | 3.04 | 898.8650506 |
| | | megaterium | | |
| 4 | g6 | Bacillus | 3.47 | 880.16544 |
| | | subtilis | | |
| 5 | g222 | Bacillus | 3.58 | 801.7634656 |
| | | mucilaginosus | | |
| 6 | g117 | Acetobacter | 2.79 | 741.6407093 |
| | | aceti | | |
| 7 | g8 | Bacillus | 2.02 | 720.2024134 |
| | | subtilis | | |
| 8 | g116 | Bacillus | 2.03 | 687.0829568 |
| | | mucilaginosus | | |
| 9 | g5 | Paenibacillus | 3.10 | 680.6038916 |
| | | azotofixans | | |
| 10 | tt30 | Bacillus | 1.97 | 552.4890136 |
| | | subtilis | | |
| 11 | g98 | Bacillus | 1.85 | 547.800975 |
| | | megaterium | | |
| 12 | b365 | Gluconobacter | 0.90 | 485.2435483 |
| | | cerinus | | |
| 13 | t98 | Bacillus | 1.87 | 475.5496708 |
| | | subtilis | | |
| 14 | g82 | Bacillus | 1.87 | 457.0543333 |
| | | subtilis | | |
| 15 | g143 | Azospirillum | 1.72 | 446.0397508 |
| | | brasilence | | |
| 16 | g137 | Bacillus | 0.73 | 391.2618184 |
| | | subtilis | | |
| 17 | g118 | Bacillus | 1.72 | 362.2451884 |
| | | mucilaginosus | | |
| 18 | g9 | Acetobacter | 1.74 | 352.9567783 |
| | | aceti | | |
| 19 | g2 | Bacillus | 1.78 | 341.579025 |
| | | subtilis | | |
| 20 | g99 | Bacillus | 1.36 | 322.1458217 |
| | | subtilis | | |
| 21 | g96 | Bacillus | 1.61 | 312.7717346 |
| | | subtilis | | |
| 22 | t143 | Bacillus | 1.64 | 291.601215 |
| | | megaterium | | |
| 23 | t107 | Bacillus | 1.50 | 281.2180729 |
| | | subtilis | | |
| 24 | tt39 | Pseudomonas | 1.63 | 276.7531374 |
| | | putida | | |
| 25 | t112 | Bacillus | 1.61 | 271.6741531 |
| | | megaterium | | |
| 26 | t106 | Pseudomonas | 1.67 | 270.1805171 |
| | | putida | | |
| 27 | g11 | Bacillus | 1.57 | 269.1305117 |
| | | subtilis | | |
| 28 | tt60 | Pseudomonas | 1.55 | 263.607615 |
| | | putida | | |
| 29 | g12 | Bacillus | 1.48 | 252.4420129 |
| | | megaterium | | |
| 30 | g24 | Bacillus | 1.54 | 250.9538833 |
| | | subtilis | | |
| 31 | g142 | Pseudomonas | 3.35 | 245.9343587 |
| | | stutzeri | | |
| 32 | tt18 | Bacillus | 1.47 | 239.7653663 |
| | | mucilaginosus | | |
| 33 | tt58 | Pseudomonas | 1.36 | 234.3764233 |
| | | putida | | |
| 34 | g52 | Bacillus | 1.34 | 217.0458917 |
| | | subtilis | | |
| 35 | g149 | Pseudomonas | 1.58 | 216.3873617 |
| | | putida | | |
| 36 | g97 | Bacillus | 1.33 | 191.0244821 |
| | | subtilis | | |
| 37 | b310 | Pseudomonas | 1.37 | 183.2365047 |
| | | putida | | |
| 38 | b308 | Pseudomonas | 0.16 | 175.4137494 |
| | | putida | | |
| 39 | b311 | Bacillus | 1.25 | 173.4871721 |
| | | mucilaginosus | | |
| 40 | t219 | Bacillus | 1.29 | 171.6095233 |
| | | subtilis | | |
| 41 | g172 | Bacillus | 1.13 | 151.715541 |
| | | subtilis | | |
| 42 | t8 | Bacillus | 1.18 | 149.26518 |
| | | megaterium | | |
| 43 | t224 | Pseudomonas | 2.10 | 149.191065 |
| | | stutzeri | | |
| 44 | t134 | Azospirillum | 1.19 | 147.4426411 |
| | | brasilence | | |
| 45 | b282 | Bacillus | 1.28 | 142.3217029 |
| | | subtilis | | |
| 46 | b259 | Bacillus | 1.26 | 142.3192729 |
| | | mucilaginosus | | |
| 47 | t197 | Bacillus | 1.39 | 139.0702389 |
| | | mucilaginosus | | |
| 48 | tt64 | Acetobacter | 1.20 | 137.78829 |
| | | aceti | | |
| 49 | t102 | Bacillus | 0.36 | 135.9925006 |
| | | megaterium | | |
| 50 | tt27 | Acetobacter | 1.18 | 133.9359332 |
| | | aceti | | |
| 51 | t22 | Bacillus | 1.03 | 128.2933129 |
| | | subtilis | | |
| 52 | tt70 | Bacillus | 1.13 | 125.3322995 |
| | | subtilis | | |
| 53 | g130 | Pseudomonas | 1.26 | 119.4878361 |
| | | putida | | |
| 54 | t103 | Bacillus | 1.05 | 108.9066489 |
| | | subtilis | | |
| 55 | t212 | Bacillus | 1.12 | 106.44129 |
| | | subtilis | | |
| 56 | b204 | Bacillus | 1.01 | 102.2928159 |
| | | mucilaginosus | | |
| 57 | b257 | Bacillus | 0.90 | 101.5101032 |
| | | subtilis | | |
| 58 | g109 | Bacillus | 0.92 | 100.0574321 |
| | | megaterium | | |
| 59 | tt15 | Bacillus | 0.84 | 96.16259898 |
| | | mucilaginosus | | |
| 60 | t100 | Bacillus | 0.76 | 94.38277464 |
| | | subtilis | | |
| 61 | g95 | Bacillus | 2.93 | 84.86010036 |
| | | subtilis | | |
| 62 | t91 | Pseudomonas | 0.88 | 82.76823 |
| | | stutzeri | | |
| 63 | tt49 | Pseudomonas | 0.71 | 82.52778393 |
| | | putida | | |
| 64 | tt47 | Bacillus | 0.90 | 77.55211107 |
| | | mucilaginosus | | |
| 65 | t108 | Bacillus | 1.02 | 75.3749064 |
| | | subtilis | | |
| 66 | tt29 | Bacillus | 0.76 | 69.84258615 |
| | | megaterium | | |
| 67 | g145 | Bacillus | 0.68 | 65.540502 |
| | | subtilis | | |
| 68 | tt59 | Bacillus | 0.53 | 64.4101875 |
| | | mucilaginosus | | |

Examples described above are better implementation schemes, in addition, the present invention can also be achieved in other ways. Any replacement without departing from the inventive concept is within the protection scope of the present invention.

1. A method of identifying whether Azotobacter secrets ammonia using nitrogen-free solid incubation media, said method comprising the steps of:
(1) mixing K₂HPO₄, MgSO₄.7H₂O, CaCl₂.2H₂O, glucose, FeSO₄.7H₂O, NaMoO₄.2H₂O, agar and distilled water together and stirring to obtain nitrogen-free incubation media; wherein, each liter of nitrogen-free incubation media comprises: K₂HPO₄ 0.5-2 g, MgSO₄.7H₂O 0.1-0.5 g, CaCl₂.2H₂O 0.05-0.15 g, glucose 7-13 g, FeSO₄.7H₂O 0.005-0.02 g, NaMoO₄.2H₂O 0.005-0.01 g, agar 10-20 g, and distilled water to balance;
(2) the preparation of double-layer plates are by sterilizing said nitrogen-free incubation media in said step 1 at 118-124° C. for 15-25 minutes, then adding 1 mL indicator bacteria solution into every 100 mL nitrogen-free incubation media when the temperature dropped to 46-48° C. after sterilization, and then pouring nitrogen-free incubation media containing indicator bacteria immediately into sterile petri dish and mixing them, followed by controlling thickness of nitrogen-free incubation media at 2.2-2.8 mm, then pouring water agar of 0.5-1 mm thickness into solidifying nitrogen-free incubation media, as-obtained double-layer plates comprise nitrogen-free solid incubation media containing bacterial of lower layer and sterile water agar of upper layer;
(3) inoculating bacteria to be tested on said nitrogen-free solid incubation media from said step 2, and then placing in incubator at 30° C. for 2-5 days;
(4) checking whether indicator bacteria of lower layer of double-layer plates has grown, if indicator bacteria growth occurs, measured Azotobacter does secrete ammonia to extracellular.

2. The method of claim 1, wherein said water agar is in said step 2 is prepared by adding 15 g agar into 1 L of distilled water; then sterilizing at 121° C. for 20 minutes, and placing under incubator at 50° C. for future using.

3. The method of claim 1, wherein said indicator bacteria in said step 2 is Escherichia coli.

4. The method of claim 1, wherein said K₂HPO₄, MgSO₄.7H₂O, CaCl₂.2H₂O, glucose, FeSO₄.7H₂O and NaMoO₄.2H₂O in said step 1 are analysis grade, and said agar is washed by distilled water before used.
